(19) United States
(12) Patent Application Publication
(10) Pub. No.: US 2026/0290587 A1
(43) Pub. Date: Sep. 24, 2026

(54) METHOD AND SYSTEM FOR CLASSIFYING AN IN-STENT RE-STENOSIS

(71) Applicant: BIOTRONIK SE & Co. KG, Berlin (DE)

(72) Inventors: Dominic WIST, Berlin (DE); Bjoern DIEM, Berlin (DE); Volker LANG, Berlin (DE)

(73) Assignee: BIOTRONIK SE & Co. KG, Berlin (DE)

(21) Appl. No.: 19/472,958

(22) PCT Filed: Mar. 28, 2024
(86) PCT No.: PCT/EP2024/058530

§ 371 Date: Oct. 6, 2025

(30) Foreign Application Priority Data
Apr. 18, 2023 (EP) 23168367.3

(51) Int. Cl. G16H 50/20; A61B 5/00; A61B 5/346; A61F 2/82

(52) U.S. Cl. CPC G16H 50/20; A61B 5/346; A61B 5/4851; A61B 5/7267; A61F 2/82

(57) ABSTRACT

A computer-implemented method for classifying an in-stent re-stenosis including the steps of providing a pre-acquired data set including ECG-data of a patient acquired by an implantable medical device, applying a machine learning algorithm to the data set including ECG-data for classification of an in-stent re-stenosis; and outputting a classification result representing a probability of an in-stent re-stenosis. Furthermore, a computer-implemented method for providing a trained machine learning algorithm configured to classify an in-stent re-stenosis, a system for classifying an in-stent re-stenosis and a system for providing a trained machine learning algorithm configured to classify an in-stent re-stenosis are also disclosed.

### CROSS-REFERENCE TO RELATED APPLICATIONS

This application is the United States National Phase under 35 U.S.C. §371 of PCT International Patent Application No. PCT/EP2024/058530, filed on Mar. 28, 2024, which claims the benefit of European Patent Application No. 23168367.3, filed on Apr. 18, 2023, the disclosures of which are hereby incorporated by reference herein in their entireties.

### TECHNICAL FIELD

The invention relates to a computer-implemented method for classifying an in-stent re-stenosis. Furthermore, the invention relates to a computer-implemented method for providing a trained machine learning algorithm configured to classify an in-stent re-stenosis. Moreover, the invention relates to a system for classifying an in-stent re-stenosis and a system for providing a trained machine learning algorithm configured to classify an in-stent re-stenosis.

### BACKGROUND

Sensors 2020, 20, 4303, Health Care Monitoring and Treatment for Coronary Artery Diseases: Challenges and Issues (Mokhalad Alghrairi, Nasri Sulaiman and Saad Mutashar) discloses that in-stent restenosis concerning the coronary artery refers to the blood clotting-caused re-narrowing of the blocked section of the artery, which is opened using a stent.

The failure rate for stents is in the range of 10% to 15%, where they do not remain open, thereby leading to about 40% of the patients with stent implantations requiring repeat procedure within one year, despite increased risk factors and the administration of expensive medicines. Hence, today stent restenosis is a significant cause of deaths globally.

Re-stenosis is usually diagnosed by exercise test with ECG recording, coronary CT angiography or invasively with cardiac catheterization. Especially, since the restenosis of a stent can be asymptomatic, examinations in regular intervals are necessary, usually by exercise tests at the physician's office.

Early detection of in-stent restenosis can be achieved by wireless passive pressure sensor monitoring, a pressure sensor using x-ray to read out the degree of restenosis and/or a 12-lead-ECG.

Exercise ECG recording, coronary CT angiography or invasively with cardiac catheterization all require the presence of the patient at the physician. Exercise ECG recording is not feasible to be done more than once or twice per year.

CCT and especially catheterization pose significant risks for the patient especially if repeated often. Wireless passive pressure sensors integrated in stents usually suffer from severe restrictions of transmission range, which would require additional active implants serving as transmission hubs.

A pressure sensor, a pressure measurement device or a pressure measurement using a catheter requires x-ray and is therefore not suited for early detection at home. A 12-lead ECG requires manual measurements which only a minority of patients can record by themselves and cannot be performed continuously of longer time spans.

The present disclosure is directed toward overcoming one or more of the above-mentioned problems, though not necessarily limited to embodiments that do.

### SUMMARY

It is therefore an object of the present invention to provide an improved method and system for detecting in-stent restenosis for post myocardial infarct patients as early as possible.

The object is solved by a computer-implemented method for classifying an in-stent re-stenosis having the features of claim 1.

Furthermore, the object is solved by a computer-implemented method for providing a trained machine learning algorithm configured to classify an in-stent re-stenosis having the features of claim 9.

Moreover, the object is solved by a system for classifying an in-stent re-stenosis having the features of claim 14 and a system for providing a trained machine learning algorithm configured to classify an in-stent re-stenosis having the features of claim 15.

Further developments and advantageous embodiments are defined in the dependent claims.

The present invention provides a computer-implemented method for classifying an in-stent re-stenosis. The method comprises providing a data set comprising ECG-data of a patient acquired by an implantable medical device, applying a machine learning algorithm to the (pre-acquired) data set comprising ECG-data for classification of an in-stent re-stenosis and outputting a classification result representing a probability of an in-stent re-stenosis.

Furthermore, the present invention provides a computer-implemented method for providing a trained machine learning algorithm configured to classify an in-stent re-stenosis.

The method comprises receiving a first training data set comprising ECG-data of a plurality of patients acquired by a respective implantable medical device, receiving a second training data set comprising a classification result representing a probability of an in-stent re-stenosis and training the machine learning algorithm by an optimization algorithm which calculates an extreme value of a loss function for classification of an in-stent re-stenosis.

Moreover, the present invention provides a system for classifying an in-stent re-stenosis comprising an implantable medical device configured to provide a data set comprising ECG-data of a patient, a computing unit configured to apply a machine learning algorithm to the (pre-acquired) data set comprising ECG-data for classification of an in-stent re-stenosis and an outputting unit configured to output a classification result representing a probability of an in-stent re-stenosis.

In addition, the present invention provides a system for providing a trained machine learning algorithm configured to classify an in-stent re-stenosis.

The system comprises a training computing unit configured to receive a first training data set comprising ECG-data of a plurality of patients acquired by a respective implantable medical device, said training computing unit being further configured to receive a second training data set comprising a classification result representing a probability of an in-stent re-stenosis, and wherein the training computing unit is configured to train the machine learning algorithm by an optimization algorithm which calculates an extreme value of a loss function for classification of an in-stent re-stenosis.

Machine learning algorithms are based on using statistical techniques to train a data processing system to perform a specific task without being explicitly programmed to do so. The goal of machine learning is to construct algorithms that can learn from data and make predictions. These algorithms create mathematical models that can be used, for example, to classify data or to solve regression type problems.

An idea of the present invention is to automatically detect in-stent restenosis for post myocardial infarct patients as early as possible. This is achieved by detecting any long-lasting changes of ECG signal which may be interpreted as an onset criterion for relevant in-stent restenosis.

The present invention further provides an advantageous use scenario for an implantable medical device in combination with vascular intervention. Using an implantable medical device such as an ICM allows for automatic and therefore more reliable daily monitoring for in-stent restenosis which enables earlier detection than with other non-automatic methods.

According to an aspect of the invention, the classification result comprises at least a first class representing a non-existence of an in-stent re-stenosis and a second class representing an existence of an in-stent re-stenosis and/or wherein the classification result comprises a numerical value indicative of an in-stent re-stenosis. Therefore, in-stent re-stenosis can be reliably detected.

According to a further aspect of the invention, if the numerical value indicative of an in-stent re-stenosis is outside a predetermined range, exceeds or falls below a predetermined threshold value, a notification is sent to a patient communication device and/or a communication device of a health care provider via a patient communication device. Said range and/or threshold value can advantageously be set according to predetermined parameters specific to the patient, e.g. based on a medical history of said patient and/or a generally normal range for similar patients.

According to a further aspect of the invention, the machine learning algorithm operates on the patient communication device or on a server of the health care provider. A battery service life of the implantable medical device can hence be preserved by outsourcing compute-intensive tasks to the patient communication device or the server of the health care provider.

According to a further aspect of the invention, the machine learning algorithm is configured to detect a change in the ECG-data, in particular a change lasting longer than a predetermined time period, indicative of an in-stent re-stenosis. Thus, short-term changes in ECG-data due to other effects do not have an influence on a detection result of the machine learning algorithm.

According to another aspect of the invention, a further data set is provided to the machine learning algorithm, said further data set comprising information on a location of a stent and/or electronic health record data of the patient, in particular an age, a sex and/or a medical history of the patient. This advantageously allows the detection of in-stent re-stenosis to be based at least in part on individual patient data, thus achieving a greater detection accuracy.

According to a further aspect of the invention, the machine learning algorithm is a deep neural network or a decision tree classifier. This allows for detecting non-linear correlations of an in-stent re-stenosis patient condition with ECG-data of a patient acquired by the implantable medical device.

According to a further aspect of the invention, the machine learning algorithm outputs a further classification result representing a degree of an in-stent restenosis. The machine learning algorithm is thus configured to output a probability as well as a severity of an in-stent restenosis.

According to a further aspect of the invention, the second training data set comprises at least a first class representing a non-existence of an in-stent re-stenosis and a second class representing an existence of an in-stent re-stenosis. Positive detections of in-stent re-stenosis can then be further examined by determining a degree of in-stent re-stenosis.

According to a further aspect of the invention, the first training data set comprises ECG-data of the plurality of patients with known stent status, in particular with and without in-stent restenosis, acquired by the implantable medical device or wherein the first training data set comprises data on a morphology of a QRS, a ST and a T wave.

Said training data thus covers a wide range of individual patient situations hence providing a balanced training data set.

According to a further aspect of the invention, the first training data set further comprises information on a location of a stent and/or electronic health record data of the patient, in particular an age, a sex and/or a medical history of the patient. This advantageously allows for an enhanced training of the machine learning algorithm for the detection of in-stent re-stenosis that is based at least in part on individual patient data, thus achieving a greater detection accuracy.

According to a further aspect of the invention, the first training data set further comprises data with respect to an ECG timestamp, in particular a location of the patient acquired by a patient communication device and/or environmental conditions at the location of the patient. The training of the machine learning algorithm can thus advantageously be further individualized hence achieving a greater detection accuracy.

The herein described features of the computer-implemented method for classifying an in-stent re-stenosis are also disclosed for the system for classifying an in-stent re-stenosis and vice versa.

Furthermore, the herein described features of the computer-implemented method for providing a trained machine learning algorithm configured to classify an in-stent re-stenosis are also disclosed for the system for providing a trained machine learning algorithm configured to classify an in-stent re-stenosis and vice versa.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

For a more complete understanding of the present invention and advantages thereof, reference is now made to the following description taken in conjunction with the accompanying drawings. The invention is explained in more detail below using exemplary embodiments, which are specified in the schematic figures of the drawings, in which:

FIG. 1 shows a flowchart of a computer-implemented method for classifying an in-stent re-stenosis according to a preferred embodiment of the invention;

FIG. 2 shows a flowchart of a computer-implemented method for providing a trained machine learning algorithm configured to classify an in-stent re-stenosis according to the preferred embodiment of the invention;

FIG. 3 shows a diagram of a system configured to classify an in-stent re-stenosis according to the preferred embodiment of the invention; and

FIG. 4 shows a diagram of a system for providing a trained machine learning algorithm configured to classify an in-stent re-stenosis according to the preferred embodiment of the invention.

### DETAILED DESCRIPTION

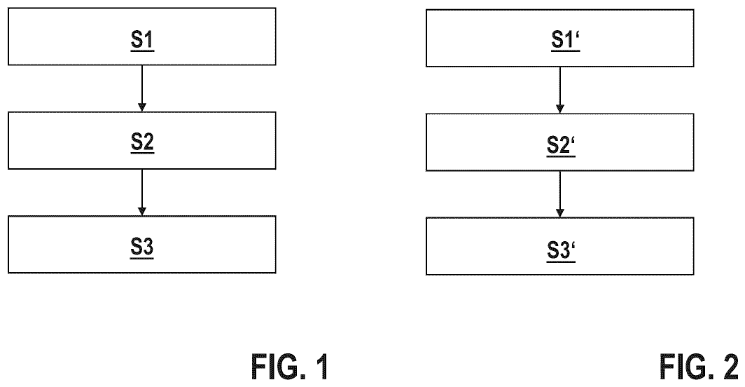

The computer-implemented method for classifying an in-stent re-stenosis shown in FIG. 1 comprises providing S1 a data set DS1 comprising ECG-data of a patient acquired by an implantable medical device 10.

Furthermore, the method comprises applying S2 a machine learning algorithm A to the (pre-acquired) data set DS1 comprising ECG-data for classification of an in-stent re-stenosis and outputting S3 a classification result 12 representing a probability of an in-stent re-stenosis.

The classification result 12 comprises at least a first class C1 representing a non-existence of an in-stent re-stenosis and a second class C2 representing an existence of an in-stent re-stenosis and/or wherein the classification result 12 comprises a numerical value indicative of an in-stent re-stenosis. Alternatively, more than two classes can be provided such that a probability of the in-stent re-stenosis can be more accurately determined.

If the numerical value indicative of an in-stent re-stenosis is outside a predetermined range, exceeds or falls below a predetermined threshold value, a notification is sent to a patient communication device 14 and/or a communication device of a health care provider 16 via a patient communication device 14.

The machine learning algorithm A operates on the patient communication device 14 or on a server 18 of the health care provider. Moreover, the machine learning algorithm A is configured to detect a change in the ECG-data, in particular a change lasting longer than a predetermined time period, indicative of an in-stent re-stenosis.

A further data set DS2 is provided to the machine learning algorithm A, said further data set DS2 comprising information on a location of a stent and/or electronic health record data of the patient, in particular an age, a sex and/or a medical history of the patient.

The machine learning algorithm A is preferably a deep neural network. Alternatively, the machine learning algorithm A can be a decision tree classifier. Moreover, the machine learning algorithm A outputs a further classification result 12 representing a degree of an in-stent restenosis.

FIG. 2 shows a flowchart of a computer-implemented method for providing a trained machine learning algorithm A configured to classify an in-stent re-stenosis according to the preferred embodiment of the invention. The method comprises receiving S1′ a first training data set TD1 comprising ECG-data of a plurality of patients acquired by a respective implantable medical device 10.

Moreover, the method comprises receiving S2′ a second training data set TD2 comprising a classification result 12 representing a probability of an in-stent re-stenosis. In addition, the method comprises training S3′ the machine learning algorithm A by an optimization algorithm which calculates an extreme value of a loss function for classification of an in-stent re-stenosis.

The second training data set TD2 comprises at least a first class C1 representing a non-existence of an in-stent re-stenosis and a second class C2 representing an existence of an in-stent re-stenosis.

The first training data set TD1 further comprises ECG-data of the plurality of patients with known stent status, in particular with and without in-stent restenosis, acquired by the implantable medical device 10 or wherein the first training data set TD1 comprises data on a morphology of a QRS, a ST and a T wave.

Moreover, the first training data set TD1 comprises information on a location of a stent and/or electronic health record data of the patient, in particular an age, a sex and/or a medical history of the patient.

The first training data set TD1 further comprises data with respect to an ECG timestamp, in particular a location of the patient acquired by a patient communication device 14 and/or environmental conditions at the location of the patient.

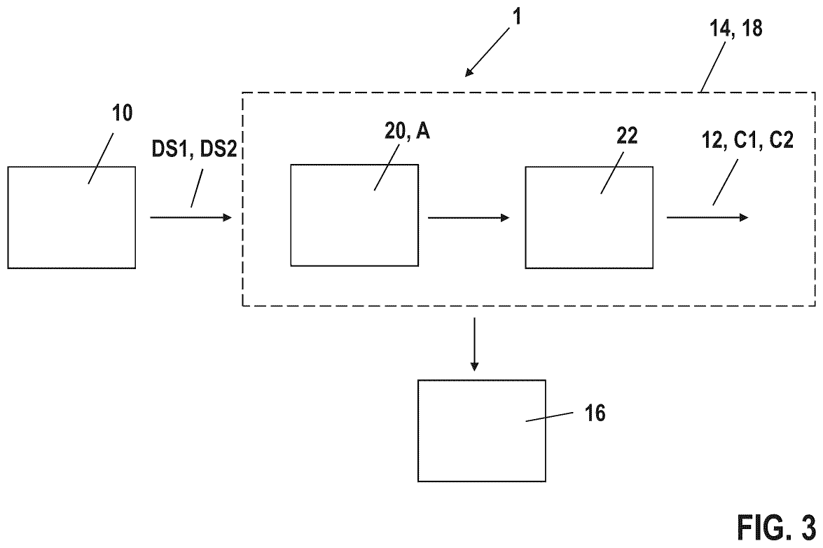

FIG. 3 shows a diagram of a system 1 configured to classify an in-stent re-stenosis according to the preferred embodiment of the invention. The system 1 comprises an implantable medical device 10 configured to provide a data set DS1 comprising ECG-data of a patient.

Furthermore, the system 1 comprises a computing unit 20 configured to apply a machine learning algorithm A to the (pre-acquired) data set DS1 comprising ECG-data for classification of an in-stent re-stenosis and an outputting unit 22 configured to output a classification result 12 representing a probability of an in-stent re-stenosis.

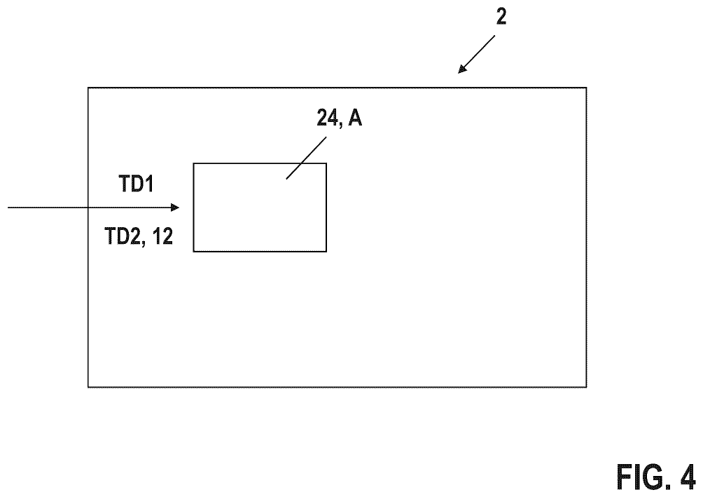

FIG. 4 shows a diagram of a system 2 for providing a trained machine learning algorithm A configured to classify an in-stent re-stenosis according to the preferred embodiment of the invention.

The system 2 comprises a training computing unit 24 configured to receive a first training data set TDI comprising ECG-data of a plurality of patients acquired by a respective implantable medical device 10, said training computing unit 24 being further configured to receive a second training data set TD2 comprising a classification result 12 representing a probability of an in-stent re-stenosis, and wherein the training computing unit 24 is configured to train the machine learning algorithm A by an optimization algorithm which calculates an extreme value of a loss function for classification of an in-stent re-stenosis.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### REFERENCE SIGNS

1 system
2 system
10 implantable medical device
12 classification result
14 patient communication device
16 communication device of a health care provider
18 server
20 computing unit
22 outputting unit
24 training computing unit
A machine learning algorithm
C1 first class
C2 second class
DS1 data set
DS2 further data set
TD1 first training data set
TD2 second training data set
S1-S3 method steps
S1′-S3′ training method steps

1. Computer-implemented method for classifying an in-stent re-stenosis comprising the steps of:
providing a pre-acquired data set comprising ECG-data of a patient acquired by an implantable medical device;
applying a machine learning algorithm to the data set comprising ECG-data for classification of an in-stent re-stenosis; and
outputting a classification result representing a probability of an in-stent re-stenosis.

2. Computer-implemented method of claim 1, wherein the classification result comprises at least a first class representing a non-existence of an in-stent re-stenosis and a second class representing an existence of an in-stent re-stenosis and/or wherein the classification result comprises a numerical value indicative of an in-stent re-stenosis.

3. Computer-implemented method of claim 2, wherein if the numerical value indicative of an in-stent re-stenosis is outside a predetermined range, exceeds or falls below a predetermined threshold value, a notification is sent to a patient communication device and/or a communication device of a health care provider via a patient communication device.

4. Computer-implemented method of claim 3, wherein the machine learning algorithm operates on the patient communication device or on a server of the health care provider.

5. Computer-implemented method of claim 1, wherein the machine learning algorithm is configured to detect a change in the ECG-data, in particular a change lasting longer than a predetermined time period, indicative of an in-stent re-stenosis.

6. Computer-implemented method of claim 1, wherein a further data set is provided to the machine learning algorithm, said further data set comprising information on a location of a stent and/or electronic health record data of the patient, in particular an age, a sex and/or a medical history of the patient.

7. Computer-implemented method of claim 1, wherein the machine learning algorithm is a deep neural network or a decision tree classifier.

8. Computer-implemented method of claim 1, wherein the machine learning algorithm outputs a further classification result representing a degree of an in-stent restenosis.

9. Computer-implemented method for providing a trained machine learning algorithm configured to classify an in-stent re-stenosis comprising the steps of:
receiving a pre-acquired first training data set comprising ECG-data of a plurality of patients acquired by a respective implantable medical device;
receiving a second training data set comprising a classification result representing a probability of an in-stent re-stenosis;
training the machine learning algorithm by an optimization algorithm which calculates an extreme value of a loss function for classification of an in-stent re-stenosis.

10. Computer-implemented method of claim 9, wherein the second training data set comprises at least a first class representing a non-existence of an in-stent re-stenosis and a second class representing an existence of an in-stent re-stenosis.

11. Computer-implemented method of claim 9, wherein the first training data set comprises ECG-data of the plurality of patients with known stent status, in particular with and without in-stent restenosis, acquired by the implantable medical device or wherein the first training data set comprises data on a morphology of a QRS, a ST and a T wave.

12. Computer-implemented method of claim 9, wherein the first training data set further comprises information on a location of a stent and/or electronic health record data of the patient, in particular an age, a sex and/or a medical history of the patient.

13. Computer-implemented method of claim 9, wherein the first training data set further comprises data with respect to an ECG timestamp, in particular a location of the patient acquired by a patient communication device and/or environmental conditions at the location of the patient.

14. System for classifying an in-stent re-stenosis comprising:
an implantable medical device configured to provide a data set comprising ECG-data of a patient;
a computing unit configured to apply a machine learning algorithm to the data set comprising ECG-data for classification of an in-stent re-stenosis; and
an outputting unit configured to output a classification result representing a probability of an in-stent re-stenosis.

15. System for providing a trained machine learning algorithm configured to classify an in-stent re-stenosis comprising:
a training computing unit configured to receive a first training data set comprising ECG-data of a plurality of patients acquired by a respective implantable medical device, said training computing unit being further configured to receive a second training data set comprising a classification result representing a probability of an in-stent re-stenosis, and wherein the training computing unit is configured to train the machine learning algorithm by an optimization algorithm which calculates an extreme value of a loss function for classification of an in-stent re-stenosis.
